(19) United States
(12) Patent Application Publication
(10) Pub. No.: US 2026/0232979 A1
(43) Pub. Date: Aug. 13, 2026

(54) Tattoo Device

(71) Applicant: Young Hee CHU, Sujeong-gu (KR)

(72) Inventor: Young Hee CHU, Sujeong-gu (KR)

(21) Appl. No.: 19/156,579

(22) PCT Filed: Feb. 2, 2024
(86) PCT No.: PCT/KR2024/001583

§ 371 Date: Aug. 14, 2025

(30) Foreign Application Priority Data
Mar. 21, 2023 (KR) 10-2023-0036455

(51) Int. Cl. A61M 37/00

(52) U.S. Cl. CPC A61M 37/0084; A61M 37/0092

(57) ABSTRACT

The present invention relates to a tattoo device, and more particularly, to a tattoo device in which a dye cartridge is replaceably housed and the dye stored therein can be delivered into the skin through a hollow needle that reciprocates.

### TECHNICAL FIELD

The present invention relates to a tattoo device and, more particularly, to a tattoo device including a replaceable dye container and configured to deliver dye into the skin through a hollow needle that reciprocates during use.

### BACKGROUND ART

In general, a tattoo device is used to create patterns or images such as symbols, letters, or shapes on human skin using tattoo dye and needles.

Conventional tattoo devices typically automate the reciprocating motion of the tattoo needle for the convenience of the operator. As the needle moves back and forth, the dye flows along the needle, and the repeated insertion of the needle into the skin at a certain depth allows the user to form a desired design on the skin in accordance with their intent.

However, these devices typically do not allow for adjustment of the exposed length of the needle, requiring users to prepare multiple needles of different lengths for different models, which is inefficient.

To address this issue, Korean Utility Model Registration No. 20-0461403 discloses a tattoo device comprising: an upper drive unit that includes a drive motor and a cam unit configured to transmit linear reciprocating motion via a link; and a needle cartridge connected to the lower end of the upper drive unit and including a tattoo needle installed to reciprocate in coordination with the link; and the needle cartridge further includes an elevation mechanism that adjusts the exposed length of the needle by vertically moving a component disposed outside the tattoo needle.

However, in this configuration, tattoo ink must be manually injected into the needle, which is inconvenient and reduces work efficiency.

Korean Patent No. 10-1623202 discloses a tattoo device including: a main body having a drive motor and a drive link configured to convert rotational force generated by the motor into linear reciprocating motion; and an ink unit that is attachable and detachable from the main body to supply ink to the needle unit.

However, in this design, the ink container is mounted laterally beside the main body, resulting in a bulky structure. Moreover, since the ink flows along the inner wall of the main body toward the needle unit, unnecessary ink loss can occur.

Korean Utility Model Registration No. 10-1831124 discloses another tattoo device in which the ink supply mechanism and the needle drive mechanism are directly connected.

However, this device includes separate motors for ink delivery and for needle reciprocation, which increases the overall weight and results in higher power consumption.

### DISCLOSURE OF THE INVENTION

### Technical Problem

The present invention has been devised to address the limitations of prior tattoo devices. Specifically, it provides a tattoo device capable of reciprocating a needle without requiring a motor, and of supplying a controlled amount of dye to the needle when needed.

### Technical Solution

To achieve the above objectives, the tattoo device according to the present invention comprises: A tattoo device comprising: an upper body portion having a dye container configured to store dye and provided with an outlet at one end, the dye container being removably accommodated in one side of the upper body portion, and a power supply unit provided at the opposite side; a center body portion disposed below the dye container and having a driving force transmission unit mounted therein, the driving force transmission unit including a tubular plunger configured to reciprocate vertically by magnetic force; a pumping unit installed between the upper body portion and the center body portion and configured to pump a predetermined amount of dye from the dye container into the plunger; a tip portion having a hollow interior, in which a needle support is housed that reciprocates together with the plunger, the needle support being configured to selectively accommodate one of a plurality of replaceable needles having the same or different numbers of needle tips, each needle being formed with a hollow therein; and a needle length adjustment unit configured to adjust the protruding length of the needle relative to the tip portion. The needle length adjustment unit includes a length adjustment case rotatably mounted between the center body portion and the tip portion, and an elevation block having a through-hole, the elevation block being rotatable together with the length adjustment case and movable upward or downward depending on the rotation direction, so as to adjust the distance between the needle support and the plunger, and deliver dye introduced into the plunger to the hollow needle.

It is preferable that the driving force transmission unit is housed within the center body portion and includes a bobbin through which the plunger penetrates and reciprocates along a central axis, and a coil wound around the periphery of the bobbin. The plunger is elongated in the longitudinal direction to extend into both the pumping unit and the needle length adjustment unit, and has a through-hole formed along its length, with a lower portion threadedly coupled to an upper interior of the elevation block. The needle length adjustment unit further comprises: a case support having an upper outer surface threadedly coupled to a lower inner surface of the center body portion, and a flange formed at a lower end of the case support with an enlarged outer diameter, the flange being configured to support an upper latching step protruding from an upper inner surface of the length adjustment case; and an elevation plate formed to match the shape of a lower inner surface of the length adjustment case, received rotatably together with the length adjustment case, and having the elevation block penetrably coupled through its center. The length adjustment case has a circular through-hole formed in its upper inner portion, and a polygonal through-hole formed in its lower inner portion with a diameter greater than that of the upper portion.

The plurality of needles may comprises: a first needle having one end fixed to the elevation block, a single needle tip connected to a central portion of the opposite end, and a first dye discharge hole formed around the shaft and in fluid communication with the hollow interior of the plunger; a second needle having one end fixed to the elevation block, three needle tips spaced apart at regular intervals around a second dye discharge hole formed at the central portion of the opposite end and in fluid communication with the hollow interior of the plunger; and a third needle having one end fixed to the elevation block, five needle tips spaced apart at regular intervals around a third dye discharge hole formed at the central portion of the opposite end and in fluid communication with the hollow interior of the plunger.

It preferable that the driving force transmission unit is housed within the center body portion and comprises: a bobbin through which the plunger is reciprocally movable along a central axis; and a coil wound around the bobbin. The plunger extends longitudinally into both the pumping unit and the needle length adjustment unit, includes a hollow interior extending along the longitudinal direction, and is formed with a lift guide slot that spirals around the lower outer surface of the plunger and communicates with the interior of the plunger. The elevation block comprises: a plunger insertion part inserted into the interior of the plunger from below, and a block body having a larger outer diameter than the plunger and positioned below the plunger. The needle length adjusting unit comprises: a guide bar that passes through the lift guide slot and couples with the plunger insertion part; a support part having a threaded upper outer surface for engagement with the lower inner surface of the center body portion and a flange portion formed at a widened lower end that supports an upper engagement step protruding from the inner surface of the upper portion of the length adjustment case; and a lift plate formed to correspond to the shape of the lower inner surface of the length adjustment case, rotatably accommodated therein together with the length adjustment case, and through which the block body is coupled centrally. The length adjustment case has an upper inner surface formed with a circular through-hole and a lower inner surface formed with a polygonal through-hole of larger diameter than the upper inner surface.

It is preferable that the needle support unit comprises: a block coupling portion connected to the elevation block; a collet portion extending longitudinally from the block coupling portion toward a distal end of the tip portion, the collet portion including a plurality of jaws spaced apart in the circumferential direction, each formed with a first tapered surface and a plurality of slits; and a clamping member that is threaded to one side of the collet portion facing the elevation block at one side, and includes a second tapered surface at the opposite side that corresponds to the first tapered surface of the jaws, the clamping member being configured to fix or release the needle depending on the rotation direction. The inner circumferential surface of a lower end of the tip portion is formed with a protruding reciprocation guide ridge in the circumferential direction, the protruding reciprocation guide ridge being internally penetrated along a central axis such that the needle and the needle support unit pass therethrough, an upper end of the protruding reciprocation guide ridge is threaded to the length adjustment case, the outer diameter of the protruding reciprocation guide ridge gradually decreases downward. The clamping member includes a ridge entry groove recessed along the circumferential direction with a width greater than the guide ridge and extending a predetermined length along an outer circumferential surface so as to allow relative movement with respect to the guide ridge.

### Advantageous Effects

The tattoo device of the present invention allows dye from a replaceable dye container to be delivered toward the needle via a plunger that reciprocates when power is applied. When necessary, a precise amount of dye can be supplied into the skin through the hollow of the needle, thereby improving operational efficiency.

Additionally, according to the present invention, the needle is reciprocated by the plunger itself, which moves back and forth under electric power. Since the dye is supplied from the dye container to the needle through the plunger by a manually operated pumping mechanism arranged between the dye container and the plunger, the overall device weight is relatively light, contributing to greater ease of use and improved work efficiency.

Furthermore, the tattoo device of the present invention is capable of generating ultrasonic vibration in the reciprocating needle, enhancing the efficiency of dye penetration into the skin.

### BRIEF DESCRIPTION OF DRAWINGS

FIG. 1 is a partially exploded perspective view of a tattoo device according to a first embodiment of the present invention.

FIG. 2 is a sectional view showing the lower portion of the tattoo device of FIG. 1.

FIG. 3 is a sectional view showing the upper portion of the tattoo device of FIG. 1.

FIG. 4 is a perspective view showing examples of needles—each having one or more needle tips—that are replaceably mounted to the distal end of the tattoo device of FIG. 1.

FIG. 5 is a perspective view illustrating a charging state of the tattoo device of FIG. 1.

FIG. 6 is a partial sectional view of a tattoo device according to a second embodiment of the present invention.

FIG. 7 is a partial sectional view of a tattoo device according to a third embodiment of the present invention.

### BEST MODE

Hereinafter, preferred embodiments of the tattoo device according to the present invention will be described in detail with reference to the accompanying drawings.

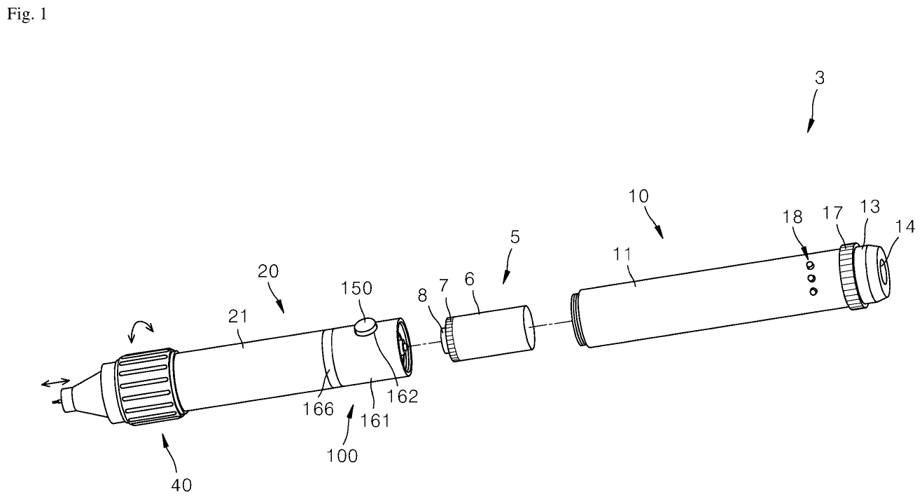

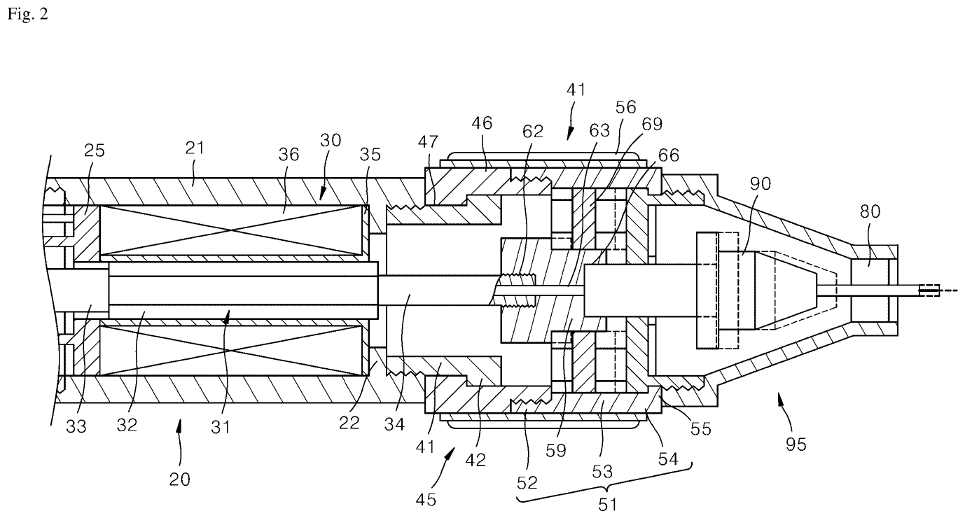

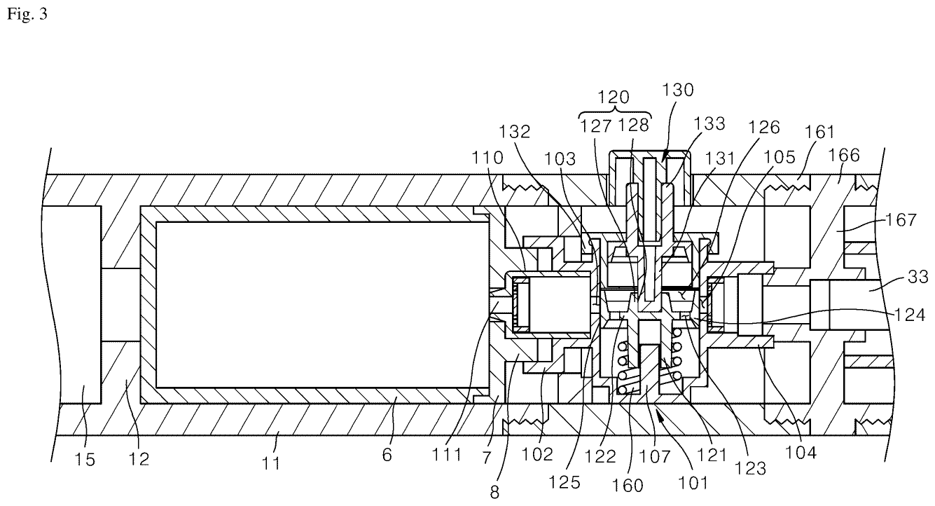

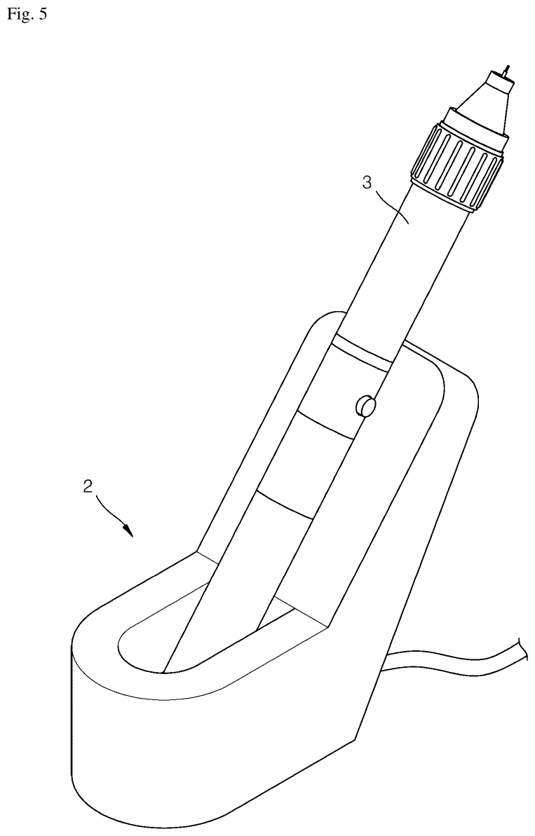

FIGS. 1 through 5 illustrate a tattoo device (3) according to a first embodiment of the present invention.

The tattoo device (3) is formed in a pen-like shape to allow easy handling by the user. The device includes: a replaceable dye container (5) for storing dye, the container being provided with an outlet (8) on one side; an upper body (10) that receives the dye container (5) in its lower portion and houses a power supply unit (15) in its upper portion; a center body (20), located below the dye container (5), in which a driving force transmission unit (30) including a tubular plunger (31) that vertically reciprocates by magnetic force is installed; a pumping unit (100), positioned between the upper body (10) and the center body (20), configured to pump a controlled amount of dye from the dye container (5) into the plunger (31); a tip portion (95), which accommodates a needle support portion (90) that ascends and descends together with the plunger (31), and in which one of a plurality of needles (71, 72, 73)—each having the same or different number of needle tips—can be replaceably mounted; and a needle length adjustment unit (40) configured to adjust the protruding length of the needles (71, 72, 73) relative to the tip portion (95).

Multiple dye containers (5) may be provided to store liquid dyes in a variety of colors, including black, white, red, orange, yellow, green, blue, purple, light green, light yellow, milk beige, and brown.

Each dye container (5) includes: a container body (6) that is open on one side to receive the dye; and a cover (7), coupled to one end of the container body (6), in which the outlet (8) is formed at the center-facing side.

The upper body (10) includes: a cylindrical upper body housing (11) that is open at both longitudinal ends and defines a first receiving space on the inside, and having a partition step (not shown) formed on the center side to support the dye container (5); and a cap (13) coupled to the upper end of the upper body housing (11).

The dye container (5) is received in the lower portion of the first receiving space, and the power supply unit (15) is received in the upper portion.

The power supply unit (15) may be implemented using a rechargeable battery. In such cases, a charging terminal (14) for the power supply unit (15) is installed at the center side of the cap (13).

Additionally, on the outer circumferential surface of the upper body housing (11), an ultrasonic control unit (17) for adjusting the operation of an ultrasonic vibrator (80), described later, and a plunger stroke speed control unit (18) for adjusting the reciprocating speed of the plunger (31) are provided. A control unit (not shown), which governs the operation of the driving force transmission unit (30) and the ultrasonic vibrator (80) in response to inputs from the ultrasonic control unit (17) and the stroke speed control unit (18), is built into one side of the upper body housing (11).

Preferably, the ultrasonic control unit (17) is implemented as a dial-type controller, as shown in the figures. The stroke speed control unit (18) is preferably formed of multiple buttons that allow stepwise adjustment of the reciprocating speed of the plunger (31).

Meanwhile, the driving force transmission unit (30) includes a bobbin (35) housed within the center body (20), and a coil (36) wound around the bobbin (35).

Although not illustrated in detail, the coil (36) is electrically connected to the power supply unit (15) via the control unit. The plunger (31) is vertically reciprocated within the bobbin (35) depending on whether current is supplied to the coil (36). The plunger (31) includes: a plunger body portion (32) having a longitudinal through-hole; a dye inlet tube (33) extending from the upper end of the plunger body portion (32) toward the upper body (10) and into the pumping unit (100); and a dye outlet tube (34) extending from the lower end of the plunger body portion (32) toward the needle length adjustment unit (100), fluidly connected to the needle support portion (90), and formed with threads on its outer lower surface.

The bobbin (35) has a polygonal inner surface that prevents the plunger body (32) from rotating, and the outer surface of the plunger body (32) is correspondingly shaped to fit this configuration.

The center body unit (20) includes: a center body housing (21), which is formed in a cylindrical shape with open ends in the longitudinal direction and has a second receiving space, with threads formed on its upper and lower inner surfaces and a support ledge (22) formed on one portion of its lower inner surface; and a coil retaining member (25), which is installed inside the center body housing (21) to support the bobbin (35) and coil (36) and has a central through-hole through which the plunger (31) passes.

The needle length adjustment unit (40) includes: a case support portion (41); a length adjustment case (45); a lifting block (59); and a lifting plate (69).

The upper outer surface of the case support portion (41) is threadedly coupled to the lower inner surface of the center body housing (21), and a rotational support flange (42) having an expanded outer diameter is formed at its lower end.

The length adjustment case (45) includes: an upper case (46); a lower case (51); and a case cover (56).

The upper case (46) is formed as a hollow cylinder, with its inner upper surface protruding to contact the inner surface of the case support portion (41) above the rotational support flange (42). A locking ridge (47) supported by the rotational flange (42) is formed in the upper case, and threads are formed on its lower outer surface.

The lower case (51) is also formed as a cylindrical through-body and includes: an upper case coupling portion (52) with inner threads for connection to the upper case (46); a lifting support portion (53) extending downward from the coupling portion and having a smaller diameter than the upper case coupling portion (52), formed polygonally to support vertical motion of the lifting plate (69); and a tip coupling portion (54) extending downward from the lifting support portion (53), having a smaller outer diameter than the lifting support portion, which defining a descent limiting shoulder (55) at the lower end of the lifting support portion (53), and formed with threads on its outer surface.

The case cover (56) is made of a stretchable rubber material and is configured to cover both the upper and lower cases (46, 51).

The lifting plate (69), housed in the lifting support portion (53), is polygonally shaped to match the internal cross-section of the lifting support portion (53), enabling it to rotate together with the length adjustment case (45). Its center is also polygonally shaped to transmit rotational force to the lifting block (59).

The lifting block (59) rotates with the length adjustment case (45) and moves vertically with the lifting plate (69) depending on the direction of rotation. As it moves, it adjusts the distance between the needle support portion (90) and the plunger (31), and delivers the dye introduced into the plunger (31) through to the hollow of the needles (71, 72, 73).

The lifting block (59) includes: a plunger insertion hole (62) that receives the lower end of the dye outlet tube (34) of the plunger (31); a communication hole (63) formed in communication with the plunger insertion hole (62) but having a smaller diameter than the dye outlet tube (34); and a needle support insertion hole (66), which is in communication with the communication hole (63), has a larger inner diameter than the communication hole, and receives one side of the needle support portion (90).

The lower outer surface of the lifting block (59) is polygonally shaped to be tightly fitted into the center of the lifting plate (69).

The upper inner surface of the tip portion (95) is threadedly engaged with the lower case (51) of the length adjustment case (45). The tip portion (95) tapers inward in both inner and outer diameters toward its lower end, and houses the needle support portion (90). Its interior is formed as a through-passage so that the needles (71, 72, 73) can advance and retract through the center of the tip.

Meanwhile, the plurality of needles (71, 72, 73) may be classified into a plurality of first needles (71), second needles (72), and third needles (73).

The first needles (71) each include a first needle body (71a), with one end removably secured to the needle support portion (90). A single needle tip (74) is formed at the opposite end of the first needle body, which protrudes from the needle support portion. Around the needle tip (74), a first dye discharge hole (71b) is formed, which communicates with the internal hollow of the device.

Each of the second needles (72) includes a second needle body (72a), with one end also detachably mounted to the needle support portion (90). A second dye discharge hole (72b), communicating with the hollow, is formed at the center of the protruding end of the second needle body. Around the second dye discharge hole (72b), three needle tips (74) are spaced at regular intervals.

Each of the third needles (73) includes a third needle body (73a), with one end removably secured to the needle support portion (90). A third dye discharge hole (73b), also in communication with the hollow, is formed at the center of the opposite end of the third needle body. Around this hole (73b), five needle tips (74) are positioned at regular intervals.

The pumping unit (100) includes: a cylinder body (101), a dye inlet guide tube (110), a vacuum packing (120), a piston (130), a button (150), a cylinder housing portion (161), and a plunger coupling section (166).

The cylinder body (101) is provided with a dye inlet pipe (102) and a dye outlet pipe (104) formed on opposing sides, each incorporating dye channels (103, 105). A guide rod (107) for guiding the vacuum packing (120) is formed at the center of the inner bottom surface.

The dye inlet guide tube (110) has one end inserted into the dye inlet pipe (102) so as to communicate with the dye channel (103), and its dye inlet port (111), which faces the discharge port (8) of the dye container (5), is selectively opened and closed by a check valve. The dye outlet pipe (104) is positioned to face the dye receiving tube (33) of the plunger (31).

The vacuum packing (120) is positioned inside the cylinder body (101) to prevent any vacuum leakage. At the center of the lower portion of its partition plate (122), which includes multiple dye flow holes (123), there is a guide tube (121) into which the guide rod (107) formed within the cylinder body (101) is inserted. On the upper side of the partition plate, a boss (127) is formed with a connection groove (128) for coupling with a connector member (131) of the piston (130).

Around the edge of the partition plate (122), a sealing rim (124) is formed to come into close contact with the inner wall of the cylinder body (101) to maintain airtightness. This sealing rim (124) has a flat sealing surface (125) that blocks the dye passage (105) of the dye outlet pipe (104), and on the opposite side, a groove (126) is formed that communicates with the dye passage (103) of the dye inlet pipe (102).

The vacuum packing (120) is installed so that it is pressed upward by a spring (160) located on the lower inner side of the cylinder body (101).

The piston (130) comprises a pumping vane portion (132), which operates inside the extension tube (141) of the cap (140), and a connecting rod member (131) that is connected to the fitting groove (128) of the vacuum packing (120). This connecting rod is formed at the center of the lower part of a partition inside the pumping vane. In addition, a connection tube (133), into which a connection pin (not shown) of the button (150) is fitted, is formed on the upper portion of the piston.

The cylinder housing portion (161) provides an internal space in which the cylinder body (101) is received. It is screw-coupled to one side of the upper body main unit (11), and includes a through-hole (162) that communicates with the internal space and allows the button (150) to move back and forth.

The plunger coupling section (166) is formed as a tubular connector that links the center body main unit (21) and the cylinder housing (161). A tubular connector bushing (167) is formed therein, into which one end of the dye outlet pipe (104) is inserted on one side and the dye receiving tube (33) of the plunger (31) is inserted on the other side.

In addition, the tattoo device (3) according to the first embodiment of the present invention is further equipped with an ultrasonic vibrator (80) mounted in the tip portion (95) to induce vibration in the needles (71, 72, 73).

The ultrasonic vibrator (80) is positioned within the tip portion (95) so that it comes into contact with the protruding portion of the needle supported by the needle support (90). Although not illustrated in detail, it is electrically connected to the control unit and the ultrasonic operation unit (17), and is configured to vibrate the needle.

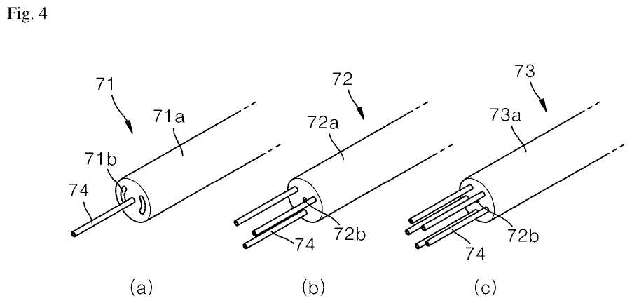

As shown in FIG. 4, the tattoo device (3) of the first embodiment can be mounted on a charging stand (2) with its charging terminal (14) facing downward, allowing for recharging.

The tattoo device (3) of the first embodiment is designed to deliver the dye stored in the replaceable dye container (5) to the needle side via the plunger (31), which moves forward and backward when powered. When needed, the device can dispense a fixed quantity of dye through the hollow needle and into the skin, enhancing work efficiency.

Furthermore, the plunger (31), which performs linear reciprocating motion during operation, drives the needle's movement, which reduces the overall weight of the device. Also, because the pumping unit (100) between the dye container (5) and the plunger (31) is manually operated, dye can be supplied efficiently to the needle via the plunger (31), further improving operational convenience and efficiency.

Additionally, the ultrasonic vibration imparted to the reciprocating needle improves the permeation efficiency of the dye into the skin.

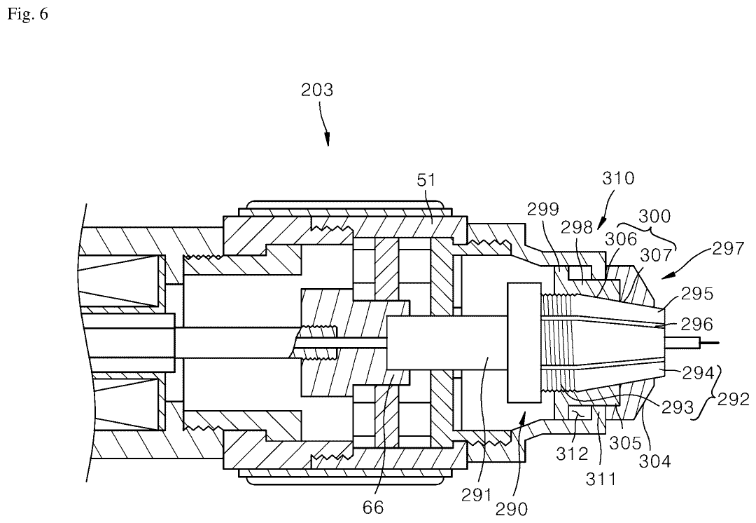

Meanwhile, FIG. 6 illustrates a tattoo device (203) according to a second embodiment of the present invention. Components that perform the same function as those described in earlier drawings are denoted with the same reference numerals.

Except for the tip portion (310) and the needle support unit (290), the tattoo device (203) of the second embodiment shares the same structure as the tattoo device (3) of the first embodiment.

The needle support unit (290) includes; a block connection portion (291) inserted into the needle support insertion hole (66) of the elevation block (59); a base coupling portion (293) having a larger outer diameter than the block connection portion (291), extending away from the elevation block (59), and having threads formed on its outer surface; and a collet portion (292), which extends longitudinally toward the front end of the tip portion (310) from the base coupling portion (293). The collet portion (292) includes a plurality of jaws (294), arranged circumferentially with gaps between them and each having a first tapered portion (295), and multiple slits (296) are formed between the jaws.

A tightening member (297) is provided, though which is passed by the collet portion (292), has one side threaded to the base coupling portion (293), and includes, on its opposite inner surface, a second tapered portion (300) corresponding to the first tapered portion (295). Depending on the rotation direction, the tightening member (297) engages or releases the needles (71, 72, 73) by compressing or relaxing the jaws (294).

The tightening member (297) includes a first unitary tightening segment (298) and a second unitary tightening segment (304). The first unitary tightening segment (298) is formed in a cylindrical shape, and on the outer surface of one end facing the elevation block (59), a descent-limiting ridge (299) protrudes along the side. The inner surface of the same end has threads for connection with the base coupling portion (293). The opposite inner surface narrows in diameter to form a first unitary taper (306). The second unitary tightening segment (304) is provided with a coupling groove (305), which is coupled to the opposite end of the first unitary tightening segment (298) with partial insertion. The one end of the coupling groove (305) is positioned at a fixed distance from the descent-limiting ridge (299) to define a step entry groove (312). Inside the coupling groove (305), the inner surface tapers further inward along the extension line of the first unitary taper (306), forming a second unitary taper (307) via a through-hole (306). The first unitary taper (306) and the second unitary taper (307) together form the second tapered portion (300).

The step entry groove (312) extends longitudinally with a width greater than that of a reciprocation guide ridge (311), described below, which is formed in the tip portion (310). This configuration allows the needle support unit (290) to move relative to the reciprocation guide ridge (311).

The tip portion (310) has an internal through-hole that allows both the needles (71, 72, 73) and the needle support unit (290) to pass through its center. Its upper end is threadedly connected to the lower case (51) of the length adjustment case (45). The outer diameter of the tip portion (310) gradually decreases toward the bottom and then extends with a fixed diameter at the lower section. On the inner surface of the lower end, the reciprocation guide ridge (311) protrudes circumferentially.

The tattoo device (203) of the second embodiment enables easier needle replacement through the collet structure, which further enhances operational efficiency.

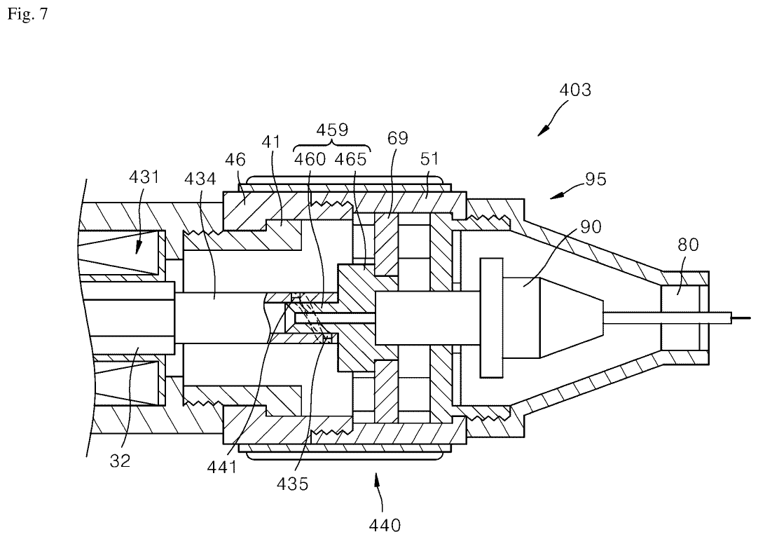

Meanwhile, FIG. 7 illustrates a tattoo device (403) according to a third embodiment of the present invention. Components that perform the same functions as in previous drawings are denoted with the same reference numerals.

The tattoo device (403) of the third embodiment shares the same structure as the tattoo device of the first embodiment, except for the plunger (431), elevation block (459), and guide bar (441).

The plunger (431) includes a plunger body portion (32), a dye inlet tube (33), and a dye outlet tube (434).

The dye outlet tube (434) has a spiral elevation guide slot (435) formed to penetrate from the lower outer surface to the interior, enabling communication between the exterior and interior.

The elevation block (459) includes a plunger insertion section (460), which is inserted into the interior of the plunger from below, and a block body (465), which has a larger outer diameter than the plunger and is positioned below it.

The needle length adjustment unit (440) further includes a guide bar (441) that passes through the helical elevation guide slot (435) and engages with the plunger insertion section (460).

The foregoing description of the present invention has been provided with reference to illustrative examples depicted in the drawings. However, these examples are merely illustrative and not intended to limit the scope of the invention in any way. It will be apparent to those skilled in the art that various modifications and equivalent alternative embodiments may be devised without departing from the spirit and scope of the invention. Therefore, the true scope of legal protection afforded by the present invention shall be defined by the appended claims and their equivalents.

1. A tattoo device comprising:
an upper body portion having a dye container configured to store dye and provided with an outlet at one end, the dye container being removably accommodated in one side of the upper body portion, and a power supply unit provided at the opposite side;
a center body portion disposed below the dye container and having a driving force transmission unit mounted therein, the driving force transmission unit including a tubular plunger configured to reciprocate vertically by magnetic force;
a pumping unit installed between the upper body portion and the center body portion and configured to pump a predetermined amount of dye from the dye container into the plunger;
a tip portion having a hollow interior, in which a needle support is housed that reciprocates together with the plunger, the needle support being configured to selectively accommodate one of a plurality of replaceable needles having the same or different numbers of needle tips, each needle being formed with a hollow therein; and
a needle length adjustment unit configured to adjust the protruding length of the needle relative to the tip portion,
wherein the needle length adjustment unit includes a length adjustment case rotatably mounted between the center body portion and the tip portion, and an elevation block having a through-hole, the elevation block being rotatable together with the length adjustment case and movable upward or downward depending on the rotation direction, so as to adjust the distance between the needle support and the plunger, and deliver dye introduced into the plunger to the hollow needle.

2. The tattoo device according to claim 1,
wherein the driving force transmission unit is housed within the center body portion and includes a bobbin through which the plunger penetrates and reciprocates along a central axis, and a coil wound around the periphery of the bobbin;
wherein the plunger is elongated in the longitudinal direction to extend into both the pumping unit and the needle length adjustment unit, and has a through-hole formed along its length, with a lower portion threadedly coupled to an upper interior of the elevation block;
wherein the needle length adjustment unit further comprises:
a case support having an upper outer surface threadedly coupled to a lower inner surface of the center body portion, and a flange formed at a lower end of the case support with an enlarged outer diameter, the flange being configured to support an upper latching step protruding from an upper inner surface of the length adjustment case; and
an elevation plate formed to match the shape of a lower inner surface of the length adjustment case, received rotatably together with the length adjustment case, and having the elevation block penetrably coupled through its center;
wherein the length adjustment case has a circular through-hole formed in its upper inner portion, and a polygonal through-hole formed in its lower inner portion with a diameter greater than that of the upper portion.

3. The tattoo device according to claim 1,
wherein the plurality of needles comprises:
a first needle having one end fixed to the elevation block, a single needle tip connected to a central portion of the opposite end, and a first dye discharge hole formed around the shaft and in fluid communication with the hollow interior of the plunger;
a second needle having one end fixed to the elevation block, three needle tips spaced apart at regular intervals around a second dye discharge hole formed at the central portion of the opposite end and in fluid communication with the hollow interior of the plunger; and
a third needle having one end fixed to the elevation block, five needle tips spaced apart at regular intervals around a third dye discharge hole formed at the central portion of the opposite end and in fluid communication with the hollow interior of the plunger.

4. The tattoo device according to claim 1,
further comprising an ultrasonic vibrator mounted to the tip portion and configured to vibrate the needle.

5. The tattoo device according to claim 1,
wherein the driving force transmission unit is housed within the center body portion and comprises:
a bobbin through which the plunger is reciprocally movable along a central axis; and
a coil wound around the bobbin,
wherein the plunger extends longitudinally into both the pumping unit and the needle length adjustment unit, includes a hollow interior extending along the longitudinal direction, and is formed with a lift guide slot that spirals around the lower outer surface of the plunger and communicates with the interior of the plunger;
wherein the elevation block comprises: a plunger insertion part inserted into the interior of the plunger from below, and a block body having a larger outer diameter than the plunger and positioned below the plunger;
wherein the needle length adjusting unit comprises:
a guide bar that passes through the lift guide slot and couples with the plunger insertion part;
a support part having a threaded upper outer surface for engagement with the lower inner surface of the center body portion and a flange portion formed at a widened lower end that supports an upper engagement step protruding from the inner surface of the upper portion of the length adjustment case; and
a lift plate formed to correspond to the shape of the lower inner surface of the length adjustment case, rotatably accommodated therein together with the length adjustment case, and through which the block body is coupled centrally;
wherein the length adjustment case has an upper inner surface formed with a circular through-hole and a lower inner surface formed with a polygonal through-hole of larger diameter than the upper inner surface.

6. The tattoo device according to claim 1,
wherein the needle support unit comprises:
a block coupling portion connected to the elevation block;
a collet portion extending longitudinally from the block coupling portion toward a distal end of the tip portion, the collet portion including a plurality of jaws spaced apart in the circumferential direction, each formed with a first tapered surface and a plurality of slits; and
a clamping member that is threaded to one side of the collet portion facing the elevation block at one side, and includes a second tapered surface at the opposite side that corresponds to the first tapered surface of the jaws, the clamping member being configured to fix or release the needle depending on the rotation direction;
wherein the inner circumferential surface of a lower end of the tip portion is formed with a protruding reciprocation guide ridge in the circumferential direction, the protruding reciprocation guide ridge being internally penetrated along a central axis such that the needle and the needle support unit pass therethrough, an upper end of the protruding reciprocation guide ridge is threaded to the length adjustment case, the outer diameter of the protruding reciprocation guide ridge gradually decreases downward;
wherein the clamping member includes a ridge entry groove recessed along the circumferential direction with a width greater than the guide ridge and extending a predetermined length along an outer circumferential surface so as to allow relative movement with respect to the guide ridge.
